(19) United States
(12) Patent Application Publication
(10) Pub. No.: US 2022/0411844 A1
(43) Pub. Date: Dec. 29, 2022

(54) METHODS OF FERMENTATION OF RECOMBINANT BACILLUS SPORES

(71) Applicant: Bayer CropScience LP, St. Louis, MO (US)

(72) Inventors: Bendy ESTIME, Allentown, PA (US); J. Antonio ROCHA-VALADEZ, Davis, CA (US)

(21) Appl. No.: 17/778,488

(22) PCT Filed: Nov. 20, 2020
(86) PCT No.: PCT/US2020/061682

§ 371 Date: May 20, 2022

Related U.S. Application Data
(60) Provisional application No. 62/939,560, filed on Nov. 22, 2019.

(51) Int. Cl. C12P 21/02; C12N 1/20; C12N 3/00; C12N 15/62; C12N 9/78

(52) U.S. Cl. CPC C12P 21/02; C12N 1/205; C12N 3/00; C12N 15/62; C12N 9/78; C12Y 305/99007; C12N 2500/32; C12N 2500/60; C12N 2500/74; C12N 2500/76; C12R 2001/075

(57) ABSTRACT

The present invention provides a method for producing a fermentation product by culturing recombinant exosporium-producing Bacillus cells that express a fusion protein of interest on the exosporium in a medium containing sources of carbon and nitrogen in a total concentration of at least 20 g/L, resulting in a fermentation broth containing a high titer of the recombinant Bacillus spores.

### REFERENCE TO RELATED APPLICATION

This application claims the benefit of U.S. Provisional Application No. 62/939,560, filed Nov. 22, 2019, which is herein incorporated by reference in its entirety.

### FIELD OF THE INVENTION

The present invention relates to the field of bacterial fermentation, and more specifically to improved fermentation media for recombinant Bacillus strains.

### REFERENCE TO SEQUENCE LISTING SUBMITTED ELECTRONICALLY

The official copy of the sequence listing is submitted electronically via EFS-Web as an ASCII-formatted sequence listing with a file named BCS199009WO_ST25.txt” created on Nov. 20, 2020, and having a size of 25 kilobytes, and is filed concurrently with the specification. The sequence listing contained in this ASCII-formatted document is part of the specification and is herein incorporated by reference in its entirety.

### BACKGROUND

Exosporium-producing Bacillus cells can be engineered to display heterologous proteins on their exosporium, using fusion proteins comprising a targeting sequence operably linked with a protein of interest. These engineered bacterial systems are useful in a variety of agricultural applications, as they can improve plant growth and/or enhance plant health, provide enhanced activity against insects, mites, nematodes and/or phytopathogens or provide herbicide tolerance properties. New methods of fermenting engineered Bacillus strains for spore display in order to improve spore titer, sporulation rates, and activity of the protein of interest are desirable for more efficient and cost-effective production of these cells and the proteins that they display. Previous methods for fermentation of these engineered bacteria were based on laboratory-scale processes that resulted in relatively low colony forming unit (CFU) counts or low rates of expression of the protein of interest. New methods of producing such engineered bacteria allow for more cost-effective use of this technology.

### SUMMARY

The present invention is directed to methods of fermenting recombinant exosporium-producing Bacillus cells capable of expressing a fusion protein comprising a protein or peptide of interest and a targeting sequence that displays the protein or peptide of interest on the exosporium of the recombinant Bacillus cells.

In one embodiment, the present invention includes a method of producing a fermentation product from recombinant exosporium-producing Bacillus cells that express a fusion protein by culturing the recombinant exosporium-producing Bacillus cells that express a fusion protein in a medium comprising
i) yeast extract at a concentration of about 2 g/L to about 30 g/L;
ii) glucose at a concentration of up to about 35 g/L; and
iii) a source of Ca′ ion.
   In such embodiment, the fusion protein comprises the protein or peptide of interest and a targeting sequence, exosporium protein, or exosporium protein fragment.

In another embodiment of this method of producing a fermentation product, the medium includes
i) yeast extract at a concentration of about 3 g/L to about 20 g/L;
ii) glucose at a concentration of up to about 35 g/L;
iii) soy flour at a concentration of up to about 50 g/L; and
iv) a source of Ca²⁺ ion.
   In one aspect, the source of Ca²⁺ ion in the above-disclosed media is CaCl₂*2H₂O. In another aspect the above-disclosed media further comprises a source of Mg′ ion. In a more particular aspect, the source of Mg²⁺ ion is MgSO₄*7H₂O.

In another embodiment, the above-disclosed media include cotton seed at a concentration of up to about 10 g/L. The above-disclosed medium may also include corn steep at a concentration of up to about 10 g/L.

In one aspect of these embodiment, pH of 6-8 is maintained during culturing in these media. pH maintenance is accomplished by addition of acid or base. Alternatively or additionally, the above-disclosed media include a buffer. In one aspect, the buffer is K₂HPO₄ and KH₂PO₄. In a more particular aspect, K₂HPO₄ is present at a concentration of at least 1 g/L and KH₂PO₄ is present at a concentration of at least 0.8 g/L.

In one embodiment of these methods, culturing occurs at 25-35° C. Additionally, culturing occurs for up to 50 hours, and/or culturing occurs until sporulation of the Bacillus cells is at least 90% complete. In one aspect of this embodiment, culturing results in a fermentation broth having a spore titer of at least 1×10⁹ spores/mL.

In another aspect, the above-disclosed media include one or more sources of carbon having a total concentration of at least 20 g/L. In another aspect, the above-disclosed media includes one or more sources of nitrogen having a total concentration of at least 3 g/L. In yet another aspect, the concentration, when combined, of the one or more sources of carbon and the one or more sources of nitrogen is at least 20 g/L.

In one embodiment, the present invention provides a method of producing a fermentation product from recombinant exosporium-producing Bacillus cells that express a fusion protein by culturing the recombinant exosporium-producing Bacillus cells that express a fusion protein in a medium that includes
a) yeast extract at a concentration of about 3 g/L to about 25 g/L, about 5 g/L to about 15 g/L, or about 10 g/L to about 15 g/L;
b) glucose at a concentration of up to about 30 g/L, about 20 g/L to about 35 g/L, or about 25 g/L to about 30 g/L;
c) soy flour at a concentration of up to about 30 g/L, about 10 g/L to about 30 g/L, or about 15 g/L to about 20 g/L; and
d) a buffer comprising K₂HPO₄ at a concentration of about 1 g/L to about 20 g/L, about 1 g/L to about 5 g/L, and KH₂PO₄ at a concentration of about 0.1 g/L to about 5 g/L, about 0.5 g/L to about 2 g/L, or about 1 g/L to about 3 g/L, or about 0.5 g/L to about 1 g/L;
e) CaCl₂*2H₂O at a concentration of about 0.010 g/L to about 1 g/L, about 0.015 g/L to about 0.80 g/L, or about 0.02 g/L to about 0.4 g/L; and
f) MgSO₄*7H₂O at a concentration of about 0.1 g/L to about 1 g/L, 0.10 g/L to about 0.80 g/L, or about 0.2 g/L to about 0.5 g/L.
   In such embodiment, the fusion protein comprises the protein or peptide of interest and a targeting sequence, exosporium protein, or exosporium protein fragment.

In a more particular aspect of this embodiment, the medium further includes about 0.01 g/L to about 0.1 g/L ZnSO₄*7H₂O.

In one aspect of the above methods, the protein or peptide of interest is a plant growth stimulating protein or peptide, a protein or peptide that protects a plant from a pathogen, and an insecticidal protein or peptide.

In another aspect, the targeting sequence, exosporium protein or exosporium protein fragment includes:

an amino acid sequence having at least about 43% identity with amino acids 20-35 of SEQ ID NO: 1, wherein the identity with amino acids 25-35 is at least about 54%;

a targeting sequence comprising amino acids 1-35 of SEQ ID NO: 1;

a targeting sequence comprising amino acids 20-35 of SEQ ID NO: 1;

a targeting sequence comprising amino acids 22-31 of SEQ ID NO: 1;

a targeting sequence comprising amino acids 22-33 of SEQ ID NO: 1;

a targeting sequence comprising amino acids 20-31 of SEQ ID NO: 1;

a targeting sequence comprising SEQ ID NO: 1; or

an exosporium protein comprising an amino acid sequence having at least 85% identity with SEQ ID NO: 2.

In another aspect, the exosporium-producing Bacillus cells are cells of a Bacillus cereus family member. In a more particular aspect, the Bacillus cereus family member is Bacillus anthracis, Bacillus cereus, Bacillus thuringiensis, Bacillus mycoides, Bacillus pseudomycoides, Bacillus samanii, Bacillus gaemokensis, Bacillus weihenstephensis, Bacillus toyoiensis and combinations thereof.

In another aspect, the recombinant exosporium-producing Bacillus cells are derived from Bacillus thuringiensis BT013A.

In one aspect of the above methods, the plant growth stimulating protein or peptide is an enzyme involved in the production or activation of a plant growth stimulating compound selected from the group consisting of an acetoin reductase, an indole-3-acetamide hydrolase, a tryptophan monooxygenase, an acetolactate synthetase, an α-acetolactate decarboxylase, a pyruvate decarboxylase, a diacetyl reductase, a butanediol dehydrogenase, an aminotransferase, a tryptophan decarboxylase, an amine oxidase, an indole-3-pyruvate decarboxylase, an indole-3-acetaldehyde dehydrogenase, a tryptophan side chain oxidase, a nitrile hydrolase, a nitrilase, a peptidase, a protease, an adenosine phosphate isopentenyltransferase, a phosphatase, an adenosine kinase, an adenine phosphoribosyltransferase, CYP735A, a 5′ribonucleotide phosphohydrolase, an adenosine nucleosidase, a zeatin cis-trans isomerase, a zeatin 0-glucosyltransferase, a β-glucosidase, a cis-hydroxylase, a CK cis-hydroxylase, a CK N-glucosyltransferase, a 2,5-ribonucleotide phosphohydrolase, an adenosine nucleosidase, a purine nucleoside phosphorylase, a zeatin reductase, a hydroxylamine reductase, a 2-oxoglutarate dioxygenase, a gibberellic 2B/3B hydrolase, a gibberellin 3-oxidase, a gibberellin 20-oxidase, a chitosanase, a chitinase, a β-1,3-glucanase, a β-1,4-glucanase, a β-1,6-glucanase, an aminocyclopropane-1-carboxylic acid deaminase, and an enzyme involved in producing a nod factor.

In another aspect, the enzyme degrades or modifies a bacterial, fungal, or plant nutrient source selected from the group consisting of a cellulase, a lipase, a lignin oxidase, a protease, a glycoside hydrolase, a phosphatase, a nitrogenase, a nuclease, an amidase, a nitrate reductase, a nitrite reductase, an amylase, an ammonia oxidase, a ligninase, a glucosidase, a phospholipase, a phytase, a pectinase, a glucanase, a sulfatase, a urease, a xylanase, and a siderophore.

In another aspect, the protein or peptide is insecticidal protein. In a more particular aspect, the insecticidal protein is a VIP insecticidal protein, an endotoxin, a Cry toxin, a protease inhibitor protein or peptide, a cysteine protease, a serine protease, and a chitinase.

In another aspect, the protein or peptide is a protein or peptide that protects a plant from a pathogen, such as a protease or a lactonase.

In one embodiment of this aspect, the serine protease has an amino acid sequence having at least 95%, at least 98%, or at least 99% identity to any one of SEQ ID NOs: 5-7.

The present invention also encompasses a fermentation broth or fermentation product that is produced by the method described above.

In yet another aspect, a fermentation broth is provided comprising: a) yeast extract at a concentration of about 3 g/L to about 25 g/L; b) glucose at a concentration of up to about 30 g/L; c) soy flour at a concentration of up to about 30 g/L; d) a buffer comprising K₂HPO₄ at a concentration of about 0.5 g/L to about 5 g/L and KH₂PO₄ at a concentration of about 0.1 g/L to about 5 g/L; e) CaCl₂*2H₂O at a concentration of about 0.010 g/L to about 1 g/L; and f) MgSO₄*7H₂O at a concentration of about 0.1 g/L to about 1.5 g/L. In certain embodiments the fermentation broth may further comprise recombinant exosporium-producing Bacillus cells that express a fusion protein, wherein the fusion protein comprises the protein or peptide of interest and a targeting sequence, exosporium protein, or exosporium protein fragment.

### BRIEF DESCRIPTION OF THE DRAWINGS

FIG. 1 shows scaled tdTomato performance in conventional yeast extract-based media and novel media prototypes M0, M2, and M5.

FIG. 2 shows the performance of novel media M2 in in several species of Bacillus from the Bacillus cereus family, including Bacillus thuringiensis.

FIG. 3 shows tdTomato fluorescence using novel media M2 in Strains #1-4, indicating robust protein expression with the use of the novel media.

FIG. 4 shows spore titer and cargo production when media M2 was used with bacterial systems displaying tdTomato.

FIG. 5 shows tdTomato performance in Base Medium and novel media prototypes M2 and OM3 at microreactor scale.

FIG. 6 shows Sep1 protease activity performance in Base Medium and novel media prototypes M2 and OM3.

### BRIEF DESCRIPTION OF THE SEQUENCES

SEQ ID NO: 1 is a BclA promoter from B. cereus.

SEQ ID NO: 2 is amino acids 1-41 of BclA (B. anthracis Sterne).

SEQ ID NO: 3 is the amino acid sequence for the tdTomato fluorescent protein.

SEQ ID NO: 4 is full length BclA (B. anthracis Sterne).

SEQ ID NO: 5 is the amino acid sequence for serine protease from Bacillus firmus DS-1 (Sep1).

SEQ ID NO: 6 is the amino acid sequence for serine protease from Bacillus firmus Strain 1 (Sep1).

SEQ ID NO: 7 is the amino acid sequence for a serine protease variant with a deletion.

SEQ ID NO: 8 is the amino acid sequence for an endoglucanase from Bacillus subtilis.

SEQ ID NO: 9 is the amino acid sequence for a phospholipase from Bacillus thuringiensis.

SEQ ID NO: 10 is the amino acid sequence for a chitosinase from Bacillus subtilis.

### DETAILED DESCRIPTION

Certain species of Bacillus produce an exosporium on the outermost layer of their endospore. Systems for engineering recombinant Bacillus cells that display fusion proteins on such exosporium have been developed. Examples of such systems are described in U.S. Pat. No. 9,133,251, International Publication Nos. WO 2014/145964 and WO 2016/044655, each of which are hereby incorporated herein by reference in their entirety. These recombinant exosporium-producing Bacillus cells are capable of expressing a fusion protein comprising a peptide and a targeting sequence, such that the peptide is targeted to and displayed on the exosporium. Bacillus exosporium display has the potential to deliver peptides or proteins of interest to plants via seed, foliar, or soil treatments. Prior to the present disclosure, it was believed that the use of media enriched in carbon and nutrient sources for the fermentation of exosporium-producing Bacillus expressing fusion protein on the exosporium led to a detrimental loss of the expressed fusion protein. Therefore, previously, a very lean media was used, which was based on yeast extract as the principal source of carbon and nitrogen. However, the present disclosure shows that media rich in carbon and nitrogen sources are highly effective, providing high activity of the protein or peptide of interest due to both higher CFU counts and higher protein or peptide expression per spore.

Therefore, the present disclosure provides improved methods of fermenting exosporium-producing bacteria engineered to display heterologous proteins on their exosporium, using a media rich in carbon and nitrogen sources. These novel fermentation methods result in improved CFU counts with retained high sporulation efficiency and higher protein activity compared with previously used media. Fermentation media described herein produce high cell density at scales ranging from 20 L to 3,000 L or more with a low cost of goods. These new methods of fermentation allow for improved use of engineered Bacillus strains at a commercially useful scale.

### I. Methods of Fermenting Recombinant Exosporium-Producing Bacillus

The present disclosure provides methods of fermenting a Bacillus strain capable of displaying a protein of interest on its exosporium by culturing the strain in the presence of the novel media provided herein. The novel fermentation media provided herein result in an improved colony forming unit count of the cultured cells and increased activity of a protein or peptide of interest displayed on the exosporium of the cells. Fermentation media provided herein may comprise one or more of yeast extract, soy flour, glucose, Ca²⁺ ion, or Mg²⁺ ion.

During fermentation, as nutrients are depleted, cells begin the transition from growth phase to sporulation phase, such that the final product of fermentation is largely spores, metabolites and residual fermentation medium. In submerged fermentation culture processes, such as those described herein, the product of the microbial culture process is referred to as a “fermentation broth.” Such broth may be concentrated, as described above. The concentrated fermentation broth may be washed, for example, via a diafiltration process, to remove residual fermentation broth and metabolites. The term “broth concentrate,” as used herein, refers to fermentation broth that has been concentrated by conventional industrial methods, as described above, but remains in liquid form. The term “fermentation product”, as used herein, refers to fermentation broth, broth concentrate and/or dried fermentation broth or broth concentrate, referred to herein as dried fermentation broth.

Fermentation media disclosed herein may comprise an enriched source of amino acids; for example yeast extract, such as Yeast Extract for Microbial Growth Medium (Sigma, St. Louis, Mo., USA), Yeast Extract Bacteriological (Thomas Scientific, Swedesboro, N.J., USA), or Yeast Extract (LabScientific, Highlands, N.J., USA). Other enriched sources of amino acids may also be used, including N-Z-AMINE A® (Sigma, St. Louis, Mo., USA), or BD Bacto Casamino Acids (BD Biosciences, San Jose, Calif., USA). In certain embodiments, yeast extract may be provided at a concentration of up to about 30 g/L, for example a concentration of between about 2 g/L to about 30 g/L, or in some embodiments a concentration of between about 5 g/L and about 10 g/L. In specific embodiments, yeast extract may be present in the disclosed media at a concentration of approximately 3 g/L, approximately 5 g/L, approximately 10 g/L, or approximately 25 g/L.

Media provided by the instant disclosure may further comprise a nutrient source capable of providing protein, vitamins, minerals, and/or carbohydrates. Nutrient sources for media used in the disclosed fermentation process may be selected from the group consisting of soy flour, peptone, nitrates, ammonium chloride, ammonium sulfate, ammonium nitrate and amino acids. Exemplary sources of these nutrients include soy flour or peptone, such as soy peptone. Nutrient sources for use in the disclosed media include, but are not limited to, Soybean Flour (Sigma, St. Louis, Mo., USA), or Peptone from GLYCINE MAX® (Sigma, St. Louis, Mo., USA). In one embodiment, the total concentration of nutrient sources may be up to about 50 g/L, up to about 30 g/L, up to about 20 g/L, up to about 15 g/L, up to about 10 g/L or between about 5 g/L to about 35 g/L, about 10 g/L to about 30 g/L, or about 10 to 25 g/L. In one embodiment, nutrient sources used in the disclosed fermentation process are selected from the group consisting of soy flour and peptone. In certain embodiments, soy flour may be present at a concentration of up to about 50 g/L, for example a concentration of between about 5 g/L to about 35 g/L, such as a concentration of about 10 g/L to about 30 g/L. In specific embodiments, soy flour may be present in the disclosed media at a concentration of approximately 10 g/L, approximately 15 g/L, approximately 20 g/L, or approximately 30 g/L.

In further embodiments, media disclosed herein comprise one or more sources of carbon and include a carbohydrate, such as glucose. Sources of carbon for media used in the disclosed fermentation process are selected from the group consisting of fructose, glucose, galactose, sucrose, lactose, mannitol, maltose, trehalose, soluble starch, molasses, sugar cane juice, and beet juice. In one embodiment a source of carbon is selected from the group consisting of fructose, glucose, galactose, sucrose, lactose, mannitol, maltose, trehalose. In another embodiment, the total concentration of the sources of carbon may be present up to about 50 g/L, up to about 40 g/L, up to about 35 g/L, up to about 30 g/L, up to about 25 g/L, up to about 20 g/L, or about 10 g/L to about 50 g/L, about 20 g/L to about 35 g/L, or about 25 g/L to about 30 g/L. In certain embodiments, the sources of carbon may be present in the disclosed media at a concentration of approximately 25 g/L, or approximately 30 g/L. In exemplary embodiments, glucose may be present at a concentration of up to about 50 g/L, up to about 40 g/L, up to about 35 g/L, up to about 30 g/L, up to about 25 g/L, up to about 20 g/L, for example at a concentration of about 10 g/L to about 50 g/L, or at a concentration of about 15 g/L to about 40 g/L or at a concentration of up to about 35 g/L, for example, at a concentration of about 20 g/L to about 35 g/L, such as a concentration of about 25 g/L to about 30 g/L. In certain embodiments, glucose may be present in the disclosed media at a concentration of approximately 25 g/L, or approximately 30 g/L.

Novel fermentation media provided herein may further comprise cotton seed flour, for example at concentrations of up to about 10 g/L, such as at approximately 2 g/L or 5 g/L or 2 g/L to 7 g/L. Cotton seed flour is a fine yellow flour made from cotton seed embryo, which is known commercially as PHARMAMEDIA® and is available from Archer Daniels Midland Company. Cotton seed flour is mainly a nitrogen source, as it is rich in protein, but it also provides some carbohydrate. Media of the present disclosure may further comprise corn steep liquor at concentrations of up to about 10 g/L, for example at approximately 2 g/L or 5 g/L or 2 g/L to 7 g/L. Corn steep liquor is a by-product of corn wet-milling and is a viscous concentrate of corn solubles, containing amino acids, vitamins and minerals.

Fermentation media disclosed herein may further comprise a buffer for regulating pH during the fermentation process. Alternatively, pH of the provided media may be controlled via the addition of acid or base. Several methods of controlling pH of a fermentation medium are known in in the art, including via buffers known in the art or via addition or acid or base if the fermentation equipment allows for this. Buffering of media provided herein is further described in Example 3 and Table 2.

Fermentation media disclosed herein may further comprise one or more source of salts of divalent cations. A source of salts of divalent cation may be selected from the group consisting of chlorides, sulfates, hydroxides, carbonates, bicarbonates, nitrates of each of Ca′, Mg²⁺ and Zn²⁺. In one embodiment, the source of salts of divalent cation may be selected from the group consisting of calcium chloride or magnesium sulfate. In one embodiment, the source of salts of divalent cation may be present in the media at a concentration of about 0.010 g/L to about 2.5 g/L, about 0.02 g/L to about 2 g/L, or about 0.010 g/L to about 1 g/L. In another embodiment, CaCl₂ is present in the media at a concentration of about 0.010 g/L to about 1 g/L, about 0.015 g/L to about 0.80 g/L, or about 0.02 g/L to about 0.4 g/L; in another embodiment, MgSO₄ is present at a concentration of about 0.1 g/L to about 1 g/L, 0.10 g/L to about 0.80 g/L, or about 0.2 g/L to about 0.5 g/L. In another embodiment, CaCl₂*2H₂O is present in the media at a concentration of about 0.010 g/L to about 1 g/L, about 0.015 g/L to about 0.80 g/L, or about 0.02 g/L to about 0.4 g/L; in another embodiment, MgSO₄*7H₂O is present at a concentration of about 0.1 g/L to about 1 g/L, 0.10 g/L to about 0.80 g/L, or about 0.2 g/L to about 0.5 g/L. In another embodiment, the source of salt further includes zinc sulfate. In one embodiment, ZnSO₄ is present at a concentration of about 0.010 g/L to about 1 g/L, about 0.015 g/L to about 0.80 g/L, or about 0.02 g/L to about 0.1 g/L. In another embodiment, ZnSO₄*7H₂O is present at a concentration of about 0.010 g/L to about 1 g/L, about 0.015 g/L to about 0.80 g/L, or about 0.02 g/L to about 0.1 g/L.

The culturing of a Bacillus strain can take place for any suitable time conducive for the cells to sporulate. For example, the culturing can take place from about 1 to about 72 hours (h), from about 5 to about 60 h, or from about 10 to about 54 h or from 24 to 48 h. In one aspect the culturing can suitably take place for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 42, 48, 54, 60 h, where any of the stated values can form an upper or lower endpoint when appropriate. In another aspect, the time for culturing can be greater than or equal to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 h. In yet another aspect, the time for culturing can be less than or equal to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 h. In still another aspect, the culturing occurs for approximately 24 to 50 hours or for approximately 45 to 70 hours.

The temperature during the culturing can be from about 20 to about 55° C. from about 25 to about 40° C., or from about 28 to about 35° C. In one aspect, the temperature during the culturing can be from about 20 to about 32° C. or from about 28 to about 40° C. In another aspect, the culturing can take place at a temperature of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55° C., where any of the stated values can form an upper or lower endpoint when appropriate. In still another aspect, the culturing can take place at a temperature greater than or equal to about, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55° C. In yet another aspect, the culturing can take place at a temperature less than or equal to about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55° C. In one aspect the culturing can occur from about 28 to about 35° C. In a further aspect, the culturing can occur at about 30° C.

The methods of the present disclosure provide significant increases in spore titer when compared with laboratory-scale media such as those described in the Examples section. In certain embodiments the methods provided herein result in spore titer in excess of 1×10⁹ spores/mL as compared with spore titers of approximately 1×10⁸ when using the Base Medium, as defined in the Examples section. In certain examples, fermentation of Bacillus strains using the novel media provided herein may result in spore titers of at least about 1×10⁸ spores/mL, at least about 1.5×10⁸ spores/mL, at least about 1×10⁹, or at least about 1.5×10⁹ spores/mL.

In embodiments provided herein, fermentation of the recombinant Bacillus strains described herein using the disclosed media resulted in sporulation rates of at least 95%. For example, fermentation of the recombinant Bacillus strains described herein using the disclosed media may result in sporulation rates of at least about 95%, at least about 96%, at least about 97%, at least about 98%, or at least about 99%.

Fermentation of engineered Bacillus using the improved media disclosed herein also resulted in improved activity of the displayed protein compared with fermentation using a lean medium, such as the Base Medium. In certain embodiments, fermentation of engineered Bacillus using the improved media disclosed herein may result in activity and/or expression of the displayed protein per mL of fermentation broth that is up to about two fold, up to about five fold, up to about ten fold, up to about 20 fold, up to about 30 fold, up to about 50 fold, up to about 100 fold more than the activity of the displayed protein from the same recombinant strain fermented in a lean medium, such as the Base Medium. Fermentation of engineered Bacillus using the novel media disclosed herein may also result in improved displayed protein yield per colony forming unit of spores that is up to about up to about two fold, up to about five fold, up to about ten fold, up to about 20 fold, up to about 30 fold, up to about 50 fold, up to about 100 fold more than the displayed protein yield per colony forming unit from the same recombinant strain fermented in a lean medium, such as the Base Medium.

### II. Recombinant Bacillus Strains

The novel media and methods disclosed herein can be used to ferment recombinant exosporium-producing Bacillus strains engineered to express fusion proteins comprising a targeting sequence and a peptide. Bacillus strains useful in the present invention include strains of any exosporium-producing species of Bacillus, such as strains from the Bacillus cereus family, including Bacillus thuringiensis.

Recombinant exosporium-producing Bacillus strains may further comprise fusion proteins comprising a targeting sequence and any peptide of interest. The fusion proteins contain a targeting sequence, an exosporium protein, or an exosporium protein fragment that targets the fusion protein to the exosporium of a Bacillus cereus family member and (a) a plant growth stimulating protein or peptide; (b) a protein or peptide that protects a plant from a pathogen or pest; (c) a protein or peptide that enhances stress resistance of a plant; (d) a plant binding protein or peptide; or (e) a plant immune system enhancer protein or peptide. When expressed in Bacillus cereus family member bacteria, these fusion proteins are targeted to the exosporium layer of the spore and are physically oriented such that the protein or peptide is displayed on the outside of the spore.

This Bacillus exosporium display (BEMD) system can be used to deliver peptides, enzymes, and other proteins to plants (e.g., to plant foliage, fruits, flowers, stems, or roots) or to a plant growth medium such as soil. Peptides, enzymes, and proteins delivered to the soil or another plant growth medium in this manner persist and exhibit activity in the soil for extended periods of time. Introduction of recombinant exosporium-producing Bacillus cells expressing the fusion proteins described herein into soil or the rhizosphere of a plant leads to a beneficial enhancement of plant growth in many different soil conditions. The use of the BEMD to create these enzymes allows them to continue to exert their beneficial results to the plant and the rhizosphere over the first months of a plant's life.

### Targeting Sequences

Bacillus is a genus of rod-shaped bacteria. The Bacillus cereus family of bacteria includes the species Bacillus anthracis, Bacillus cereus, Bacillus thuringiensis, Bacillus mycoides, Bacillus pseudomycoides, Bacillus samanii, Bacillus gaemokensis, Bacillus toyoiensis, and Bacillus weihenstephensis. Under stressful environmental conditions, Bacillus cereus family bacteria undergo sporulation and form oval endospores that can stay dormant for extended periods of time. The outermost layer of the endospores is known as the exosporium and comprises a basal layer surrounded by an external nap of hair-like projections. Filaments on the hair-like nap are predominantly formed by the collagen-like glycoprotein BclA, while the basal layer is comprised of a number of different proteins. Another collagen-related protein, BclB, is also present in the exosporium and exposed on endospores of Bacillus cereus family members.

BclA, the major constituent of the surface nap, has been shown to be attached to the exosporium with its amino-terminus (N-terminus) positioned at the basal layer and its carboxy-terminus (C-terminus) extending outward from the spore.

It was previously discovered that certain sequences from the N-terminal regions of BclA and BclB could be used to target a peptide or protein to the exosporium of a Bacillus cereus endospore (see U.S. Patent Application Publication Nos. 2010/0233124 and 2011/0281316, and Thompson, et al., “Targeting of the BclA and BclB Proteins to the Bacillus anthracis Spore Surface,” Molecular Microbiology, 70(2):421-34 (2008), the entirety of each of which is hereby incorporated by reference). It was also found that the BetA/BAS3290 protein of Bacillus anthracis localized to the exosporium.

Targeting of a protein of interest (e.g., an enzyme) to the exosporium proteins can be achieved using the following motif, which may be present in a targeting sequence, exosporium protein, or exosporium protein fragment that targets the fusion protein to the exosporium of the recombinant Bacillus bacterium and comprises the sequence X₁-X₂-X₃-X₄-X₅-X₆-X₇-X₈-X₉-X₁₀-X₁₁-X₁₂-X₁₃-X₁₄-X₁₅-X₁₆, wherein:

X₁ is any amino acid or absent;

X₂ is phenylalanine (F), leucine (L), isoleucine (I), or methionine (M);

X₃ is any amino acid;

X₄ is proline (P) or serine (S);

X₅ is any amino acid;

X₆ is leucine (L), asparagine (N), serine (S), or isoleucine (I);

X₇ is valine (V) or isoleucine (I);

X₈ is glycine (G);

X₉ is proline (P);

X₁₀ is threonine (T) or proline (P);

X₁₁ is leucine (L) or phenylalanine (F);

X₁₂ is proline (P);

X₁₃ is any amino acid;

X₁₄ is any amino acid;

X₁₅ is proline (P), glutamine (Q), or threonine (T); and

X₁₆ is proline (P), threonine (T), or serine (S).

In particular, amino acids 20-35 of BclA from Bacillus anthracis Sterne strain have been found to be sufficient for targeting to the exosporium.

Any portion of BclA which includes amino acids 20-35 can be used as the targeting sequence. In addition, full-length exosporium proteins or exosporium protein fragments can be used for targeting the fusion proteins to the exosporium. Thus, full-length BclA or a fragment of BclA that includes amino acids 20-35 can be used for targeting to the exosporium.

The targeting sequence can comprise amino acids 1-35 of SEQ ID NO: 2, amino acids 20-35 of SEQ ID NO: 2, a methionine linked to amino acids 20-35 of SEQ ID NO: 2, SEQ ID NO: 2, amino acids 22-31 of SEQ ID NO: 2, amino acids 22-33 of SEQ ID NO: 2, amino acids 20-31 of SEQ ID NO: 2. Alternatively, the targeting sequence can consist of amino acids 1-35 of SEQ ID NO: 2, amino acids 20-35 of SEQ ID NO: 2, or SEQ ID NO: 2. Alternatively, the targeting sequence can consist of amino acids 22-31 of SEQ ID NO: 2, amino acids 22-33 of SEQ ID NO: 2, or amino acids 20-31 of SEQ ID NO: 2. Alternatively, the exosporium protein can comprise full length BclA (SEQ ID NO: 4), or the exosporium protein fragment can comprise a midsized fragment of BclA that lacks the carboxy-terminus, such as amino acids 1-196 of SEQ ID NO: 4.

### Fusion Proteins

The fusion proteins can comprise a targeting sequence, an exosporium protein, or an exosporium protein fragment, and at least one plant growth stimulating protein or peptide. The plant growth stimulating protein or peptide can comprise a peptide hormone, a non-hormone peptide, an enzyme involved in the production or activation of a plant growth stimulating compound or an enzyme that degrades or modifies a bacterial, fungal, or plant nutrient source. The targeting sequence, exosporium protein, or exosporium protein fragment can be any of the targeting sequences, exosporium proteins, or exosporium protein fragments described above.

The fusion proteins can comprise a targeting sequence, an exosporium protein, or an exosporium protein fragment, and at least one protein or peptide that protects a plant from a pathogen. The targeting sequence, exosporium protein, or exosporium protein fragment can be any of the targeting sequences, exosporium proteins, or exosporium protein fragments described above.

The fusion protein can be made using standard cloning and molecular biology methods known in the art. For example, a gene encoding a protein or peptide (e.g., a gene encoding a plant growth stimulating protein or peptide) can be amplified by polymerase chain reaction (PCR) and ligated to DNA coding for any of the above-described targeting sequences to form a DNA molecule that encodes the fusion protein. The DNA molecule encoding the fusion protein can be cloned into any suitable vector, for example a plasmid vector. The vector suitably comprises a multiple cloning site into which the DNA molecule encoding the fusion protein can be easily inserted. The vector also suitably contains a selectable marker, such as an antibiotic resistance gene, such that bacteria transformed, transfected, or mated with the vector can be readily identified and isolated. Where the vector is a plasmid, the plasmid suitably also comprises an origin of replication. The DNA encoding the fusion protein is suitably under the control of a sporulation promoter which will cause expression of the fusion protein on the exosporium of a B. cereus family member endospore (e.g., a native bclA promoter from a B. cereus family member). Alternatively, DNA coding for the fusion protein can be integrated into the chromosomal DNA of the B. cereus family member host.

The fusion protein can also comprise additional polypeptide sequences that are not part of the targeting sequence, exosporium protein, exosporium protein fragment, or the plant growth stimulating protein or peptide, the protein or peptide that protects a plant from a pathogen, the protein or peptide that enhances stress resistance in a plant, or the plant binding protein or peptide. For example, the fusion protein can include tags or markers to facilitate purification or visualization of the fusion protein (e.g., a polyhistidine tag or a fluorescent protein such as GFP or YFP) or visualization of recombinant exosporium-producing Bacillus cells spores expressing the fusion protein.

Expression of fusion proteins on the exosporium using the targeting sequences, exosporium proteins, and exosporium protein fragments described herein is enhanced due to a lack of secondary structure in the amino-termini of these sequences, which allows for native folding of the fused proteins and retention of activity. Proper folding can be further enhanced by the inclusion of a short amino acid linker between the targeting sequence, exosporium protein, exosporium protein fragment, and the fusion partner protein.

### Plant Growth Stimulating Proteins and Peptides

As noted above, the fusion proteins can comprise a targeting sequence, exosporium protein, or exosporium protein fragment and at least one plant growth stimulating protein or peptide. For example, the plant growth stimulating protein or peptide can comprise a peptide hormone, a non-hormone peptide, an enzyme involved in the production or activation of a plant growth stimulating compound, or an enzyme that degrades or modifies a bacterial, fungal, or plant nutrient source.

For example, where the plant growth stimulating protein or peptide comprises a peptide hormone, the peptide hormone can comprise a phytosulfokine (e.g., phytosulfokine-α), clavata 3 (CLV3), systemin, ZmlGF, or a SCR/SP11.

Where the plant growth stimulating protein or peptide comprises a non-hormone peptide, the non-hormone peptide can comprise a RKN 16D10, Hg-Syv46, an eNOD40 peptide, melittin, mastoparan, Mas7, RHPP, POLARIS, or kunitz trypsin inhibitor (KTI).

The plant growth stimulating protein or peptide can comprise an enzyme involved in the production or activation of a plant growth stimulating compound. The enzyme involved in the production or activation of a plant growth stimulating compound can be any enzyme that catalyzes any step in a biological synthesis pathway for a compound that stimulates plant growth or alters plant structure, or any enzyme that catalyzes the conversion of an inactive or less active derivative of a compound that stimulates plant growth or alters plant structure into an active or more active form of the compound.

The plant growth stimulating compound can comprise a compound produced by bacteria or fungi in the rhizosphere, e.g., 2,3-butanediol.

Alternatively, the plant growth stimulating compound can comprise a plant growth hormone, e.g., a cytokinin or a cytokinin derivative, ethylene, an auxin or an auxin derivative, a gibberellic acid or a gibberellic acid derivative, abscisic acid or an abscisic acid derivative, or a jasmonic acid or a jasmonic acid derivative.

Where the plant growth stimulating compound comprises a cytokinin or a cytokinin derivative, the cytokinin or the cytokinin derivative can comprise kinetin, cis-zeatin, trans-zeatin, 6-benzylaminopurine, dihydroxyzeatin, N6-(D2-isopentenyl) adenine, ribosylzeatin, N6-(D2-isopentenyl) adenosine, 2-methylthio-cis-ribosylzeatin, cis-ribosylzeatin, trans-ribosylzeatin, 2-methylthio-trans-ribosylzeatin, ribosylzeatin-5-monosphosphate, N6-methylaminopurine, N6-dimethylaminopurine, 2′-deoxyzeatin riboside, 4-hydroxy-3-methyl-trans-2-butenylaminopurine, ortho-topolin, meta-topolin, benzyladenine, ortho-methyltopolin, meta-methyltopolin, or a combination thereof.

Where the plant growth stimulating compound comprises an auxin or an auxin derivative, the auxin or the auxin derivative can comprise an active auxin, an inactive auxin, a conjugated auxin, a naturally occurring auxin, or a synthetic auxin, or a combination thereof. For example, the auxin or auxin derivative can comprise indole-3-acetic acid, indole-3-pyruvic acid, indole-3-acetaldoxime, indole-3-acetamide, indole-3-acetonitrile, indole-3-ethanol, indole-3-pyruvate, indole-3-acetaldoxime, indole-3-butyric acid, a phenylacetic acid, 4-chloroindole-3-acetic acid, a glucose-conjugated auxin, or a combination thereof.

The enzyme involved in the production or activation of a plant growth stimulating compound can comprise an acetoin reductase, an indole-3-acetamide hydrolase, a tryptophan monooxygenase, an acetolactate synthetase, an α-acetolactate decarboxylase, a pyruvate decarboxylase, a diacetyl reductase, a butanediol dehydrogenase, an aminotransferase (e.g., tryptophan aminotransferase), a tryptophan decarboxylase, an amine oxidase, an indole-3-pyruvate decarboxylase, an indole-3-acetaldehyde dehydrogenase, a tryptophan side chain oxidase, a nitrile hydrolase, a nitrilase, a peptidase, a protease, an adenosine phosphate isopentenyltransferase, a phosphatase, an adenosine kinase, an adenine phosphoribosyltransferase, CYP735A, a 5′ ribonucleotide phosphohydrolase, an adenosine nucleosidase, a zeatin cis-trans isomerase, a zeatin 0-glucosyltransferase, a β-glucosidase, a cis-hydroxylase, a CK cis-hydroxylase, a CK N-glucosyltransferase, a 2,5-ribonucleotide phosphohydrolase, an adenosine nucleosidase, a purine nucleoside phosphorylase, a zeatin reductase, a hydroxylamine reductase, a 2-oxoglutarate dioxygenase, a gibberellic 2B/3B hydrolase, a gibberellin 3-oxidase, a gibberellin 20-oxidase, a chitosinase, a chitinase, a β-1,3-glucanase, a β-1,4-glucanase, a β-1,6-glucanase, an aminocyclopropane-1-carboxylic acid deaminase, or an enzyme involved in producing a nod factor (e.g., nodA, nodB, or nodI).

Where the enzyme comprises a protease or peptidase, the protease or peptidase can be a protease or peptidase that cleaves proteins, peptides, proproteins, or preproproteins to create a bioactive peptide. The bioactive peptide can be any peptide that exerts a biological activity.

Examples of bioactive peptides include RKN 16D10 and RHPP.

The protease or peptidase that cleaves proteins, peptides, proproteins, or preproproteins to create a bioactive peptide can comprise subtilisin, an acid protease, an alkaline protease, a proteinase, an endopeptidase, an exopeptidase, thermolysin, papain, pepsin, trypsin, pronase, a carboxylase, a serine protease, a glutamic protease, an aspartate protease, a cysteine protease, a threonine protease, or a metalloprotease.

The protease or peptidase can cleave proteins in a protein-rich meal (e.g., soybean meal or yeast extract).

The plant growth stimulating protein can also comprise an enzyme that degrades or modifies a bacterial, fungal, or plant nutrient source. Such enzymes include cellulases, lipases, lignin oxidases, proteases, glycoside hydrolases, phosphatases, nitrogenases, nucleases, amidases, nitrate reductases, nitrite reductases, amylases, ammonia oxidases, ligninases, glucosidases, phospholipases, phytases, pectinases, glucanases, sulfatases, ureases, and xylanases. Where the enzyme is a pectinase, the enzyme may be a pectin lyase, also referred to as pectolyase, a pectate lyase, or a polygalacturonase, including an endopolygalacturonase or an exopolygalacturonase. When introduced into a plant growth medium or applied to a plant, seed, or an area surrounding a plant or a plant seed, fusion proteins comprising enzymes that degrade or modify a bacterial, fungal, or plant nutrient source can aid in the processing of nutrients in the vicinity of the plant and result in enhanced uptake of nutrients by the plant or by beneficial bacteria or fungi in the vicinity of the plant. In one embodiment, the phospholipase comprises an amino acid sequence having at least 85%, at least 90%, at least 95%, at least 96%, at least 97%, at least 98%, or at least 99% identity with SEQ ID NO: 9.

Suitable cellulases include endocellulases (e.g., an endogluconase such as a Bacillus subtilis endoglucanase, a Bacillus thuringiensis endoglucanase, a Bacillus cereus endoglucanase, or a Bacillus clausii endoglucanase), exocellulases (e.g., a Trichoderma reesei exocellulase), and β-glucosidases (e.g., a Bacillus subtilis β-glucosidase, a Bacillus thuringiensis β-glucosidase, a Bacillus cereus β-glucosidase, or a Bacillus clausii B-glucosidase).

The lipase can comprise a Bacillus subtilis lipase, a Bacillus thuringiensis lipase, a Bacillus cereus lipase, or a Bacillus clausii lipase.

In one embodiment, the lipase comprises a Bacillus subtilis lipase.

In another embodiment, the cellulase is a Bacillus subtilis endoglucanase. In one embodiment, the endoglucanase comprises an amino acid sequence having at least 85%, at least 90%, at least 95%, at least 96%, at least 97%, at least 98%, or at least 99% identity with SEQ ID NO: 8.

In yet another embodiment, the fusion protein comprises an E. coli protease PtrB.

Suitable lignin oxidases comprise lignin peroxidases, laccases, glyoxal oxidases, ligninases, and manganese peroxidases.

The protease can comprise a subtilisin, an acid protease, an alkaline protease, a proteinase, a peptidase, an endopeptidase, an exopeptidase, a thermolysin, a papain, a pepsin, a trypsin, a pronase, a carboxylase, a serine protease, a glutamic protease, an aspartate protease, a cysteine protease, a threonine protease, or a metalloprotease.

The phosphatase can comprise a phosphoric monoester hydrolase, a phosphomonoesterase (e.g., PhoA4), a phosphoric diester hydrolase, a phosphodiesterase, a triphosphoric monoester hydrolase, a phosphoryl anhydride hydrolase, a pyrophosphatase, a phytase (e.g., Bacillus subtilis EE148 phytase or Bacillus thuringiensis BT013A phytase), a trimetaphosphatase, or a triphosphatase.

Proteins and Peptides that Protects Plants from Pathogens

The fusion proteins can comprise a targeting sequence, exosporium protein, or exosporium protein fragment, and at least one protein or peptide that protects a plant from a pathogen.

The protein or peptide can comprise a protein or peptide that stimulates a plant immune response. For example, the protein or peptide that stimulates a plant immune response can comprise a plant immune system enhancer protein or peptide. The plant immune system enhancer protein or peptide can be any protein or peptide that has a beneficial effect on the immune system of a plant. Suitable plant immune system enhancer proteins and peptides include harpins, α-elastins, β-elastins, systemins, phenylalanine ammonia-lyase, elicitins, defensins, cryptogeins, flagellin proteins, and flagellin peptides (e.g., flg22).

Alternatively, the protein or peptide that protects a plant from a pathogen can be a protein or peptide that has antibacterial activity, antifungal activity, or both antibacterial and antifungal activity. Examples of such proteins and peptides include bacteriocins, lysozymes, lysozyme peptides (e.g., LysM), siderophores, non-ribosomal active peptides, conalbumins, albumins, lactoferrins, lactoferrin peptides (e.g., LfcinB), streptavidin and TasA.

The protein or peptide that protects a plant from a pathogen can also be a protein or peptide that has insecticidal activity, helminthicidal activity, suppresses insect or worm predation, or a combination thereof. For example, the protein or peptide that protects a plant from a pathogen can comprise an insecticidal bacterial toxin (e.g., a VIP insecticidal protein), an endotoxin, a Cry toxin (e.g., a Cry toxin from Bacillus thuringiensis), a protease inhibitor protein or peptide (e.g., a trypsin inhibitor or an arrowhead protease inhibitor), a cysteine protease, or a chitinase. Where the Cry toxin is a Cry toxin from Bacillus thuringiensis, the Cry toxin can be a Cry5B protein or a Cry21A protein. Cry5B and Cry21A have both insecticidal and nematocidal activity.

The protein that protects a plant from a pathogen can comprise an enzyme. Suitable enzymes include proteases and lactonases. The proteases and lactonases can be specific for a bacterial signaling molecule (e.g., a bacterial lactone homoserine signaling molecule).

Where the enzyme is a protease, the enzyme may be a serine protease, for example Sep1. Serine proteases are one of the largest and mostly widely distributed class of proteases. Serine proteases cleave peptide bonds at serine residues within a specific recognition site in a protein. These proteases are frequently used by bacteria for nutrient scavenging in the environment. Serine proteases have also been shown to exhibit nematicidal activity through digestion of intestinal tissue in nematodes. Studies of Bacillus firmus strain DS-1, which shows nematicidal activity against Meloidogyne incognita and soybean cyst nematode, revealed that the serine protease produced by that strain has serine protease activity and degraded the intestinal tissues of nematodes. Geng, C., et al., “A Novel Serine Protease, Sep1, from Bacillus firmus DS-1 Has Nematicidal Activity and Degrades Multiple Intestinal-Associated Nematode Proteins”, Scientific Reports, 2016, vol. 6, no. 25012.

In Table 1, SEQ ID NOs: 5-7 are amino acid sequences for wild-type enzymes and a variant enzyme that exhibit or are predicted to exhibit serine protease activity. Thus, for example, SEQ ID NOs: 5 and 6 provide the amino acid sequence for wild-type serine protease enzymes from two different Bacillus firmus strains and have 98% sequence similarity. SEQ ID NO: 7 provides the amino acid sequence for the same enzyme as in SEQ ID NO: 5, except for a deletion of amino acids 181-240 of SEQ ID NO: 5, such that SEQ ID NOs: 5 and 7 have 81% sequence similarity. The catalytic residues referenced in Geng, et al., 2016, above, are maintained in the variant serine protease amino acid sequence of SEQ ID NO: 7.

TABLE 1
Amino Acid Sequences for Serine Protease and Variant

| Enzyme | SEQ ID NO: |
|---|---|
| Serine Protease from Bacillus firmus DS-1 (Sep1) | 5 |
| Serine Protease from Bacillus firmus Strain 1 (Sep1) | 6 |
| Serine Protease Variant with Deletion | 7 |

Where the enzyme is a lactonase, the lactonase can comprise 1,4-lactonase, 2-pyrone-4,6-dicarboxylate lactonase, 3-oxoadipate enol-lactonase, actinomycin lactonase, deoxylimonate A-ring-lactonase, gluconolactonase L-rhamnono-1,4-lactonase, limonin-D-ring-lactonase, steroid-lactonase, triacetate-lactonase, or xylono-1,4-lactonase.

The enzyme can also be an enzyme that is specific for a cellular component of a bacterium or fungus. For example, the enzyme can comprise a β-1,3-glucanase, a β-1,4-glucanase, a β-1,6-glucanase, a chitosinase, a chitinase, a chitosinase-like enzyme, a lyticase, a peptidase, a proteinase, a protease (e.g., an alkaline protease, an acid protease, or a neutral protease), a mutanolysin, a stapholysin, or a lysozyme. In one embodiment, the chitosinase comprises an amino acid sequence having at least 85%, at least 90%, at least 95%, at least 96%, at least 97%, at least 98%, or at least 99% identity with SEQ ID NO: 10.

Proteins and Peptides that Enhance Stress Resistance in Plants

The fusion proteins can comprise a targeting sequence, exosporium protein, or exosporium protein fragment and at least one protein or peptide that enhances stress resistance in a plant.

For example, the protein or peptide that enhances stress resistance in a plant comprises an enzyme that degrades a stress-related compound. Stress-related compounds include, but are not limited to, aminocyclopropane-1-carboxylic acid (ACC), reactive oxygen species, nitric oxide, oxylipins, and phenolics. Specific reactive oxygen species include hydroxyl, hydrogen peroxide, oxygen, and superoxide. The enzyme that degrades a stress-related compound can comprise a superoxide dismutase, an oxidase, a catalase, an aminocyclopropane-1-carboxylic acid deaminase, a peroxidase, an antioxidant enzyme, or an antioxidant peptide.

The protein or peptide that enhances stress resistance in a plant can also comprise a protein or peptide that protects a plant from an environmental stress. The environmental stress can comprise, for example, drought, flood, heat, freezing, salt, heavy metals, low pH, high pH, or a combination thereof. For instance, the protein or peptide that protects a plant from an environmental stress can comprises an ice nucleation protein, a prolinase, a phenylalanine ammonia lyase, an isochorismate synthase, an isochorismate pyruvate lyase, or a choline dehydrogenase.

### Plant Binding Proteins and Peptides

The fusion proteins can comprise a targeting sequence, exosporium protein, or exosporium protein fragment and at least plant binding protein or peptide. The plant binding protein or peptide can be any protein or peptide that is capable of specifically or non-specifically binding to any part of a plant (e.g., a plant root or an aerial portion of a plant such as a leaf, stem, flower, or fruit) or to plant matter. Thus, for example, the plant binding protein or peptide can be a root binding protein or peptide, or a leaf binding protein or peptide.

Suitable plant binding proteins and peptides include adhesins (e.g., rhicadhesin), flagellins, omptins, lectins, expansins, biofilm structural proteins (e.g., TasA or YuaB) pilus proteins, curlus proteins, intimins, invasins, agglutinins, and afimbrial proteins.

Recombinant Bacillus that Express the Fusion Proteins

The fusion proteins described herein can be expressed by recombinant exosporium-producing Bacillus cells. The fusion protein can be any of the fusion proteins discussed above.

The recombinant exosporium-producing Bacillus cells can coexpress two or more of any of the fusion proteins discussed above. For example, the recombinant exosporium-producing Bacillus cells can coexpress at least one fusion protein that comprises a plant binding protein or peptide, together with at least one fusion protein comprising a plant growth stimulating protein or peptide, at least one fusion protein comprising a protein or peptide that protects a plant from a pathogen, or at least one protein or peptide that enhances stress resistance in a plant.

The recombinant exosporium-producing Bacillus cells can comprise Bacillus anthracis, Bacillus cereus, Bacillus thuringiensis, Bacillus mycoides, Bacillus pseudomycoides, Bacillus samanii, Bacillus gaemokensis, Bacillus weihenstephensis, Bacillus toyoiensis or a combination thereof. For example, the recombinant exosporium-producing Bacillus cells can comprise Bacillus cereus, Bacillus thuringiensis, Bacillus pseudomycoides, or Bacillus mycoides. In particular, the recombinant exosporium-producing Bacillus cells can comprise Bacillus thuringiensis or Bacillus mycoides.

To generate recombinant exosporium-producing Bacillus cells expressing a fusion protein, any Bacillus cereus family member can be conjugated, transduced, or transformed with a vector encoding the fusion protein using standard methods known in the art (e.g., by electroporation). The bacteria can then be screened to identify transformants by any method known in the art. For example, where the vector includes an antibiotic resistance gene, the bacteria can be screened for antibiotic resistance. Alternatively, DNA encoding the fusion protein can be integrated into the chromosomal DNA of a B. cereus family member host. The recombinant exosporium-producing Bacillus cells can then exposed to conditions which will induce sporulation. Suitable conditions for inducing sporulation are known in the art. For example, the recombinant exosporium-producing Bacillus cells can be plated onto agar plates, and incubated at a temperature of about 30° C. for several days (e.g., 3 days).

Inactivated strains, non-toxic strains, or genetically manipulated strains of any of the above species can also suitably be used. For example, a Bacillus thuringiensis that lacks the Cry toxin can be used. Alternatively or in addition, once the recombinant B. cereus family spores expressing the fusion protein have been generated, they can be inactivated to prevent further germination once in use. Any method for inactivating bacterial spores that is known in the art can be used. Suitable methods include, without limitation, heat treatment, gamma irradiation, x-ray irradiation, UV-A irradiation, UV-B irradiation, chemical treatment (e.g., treatment with gluteraldehyde, formaldehyde, hydrogen peroxide, acetic acid, bleach, or any combination thereof), or a combination thereof. Alternatively, spores derived from nontoxigenic strains, or genetically or physically inactivated strains, can be used.

### III. Scalability

The novel media of the present invention may be used for fermentation in any suitable vessel, including glass or plastic tubes or microtiter plates, glass or stainless steel flasks, bottles, or carboys, microreactors, or bioreactors. Bioreactors suitable for use with the disclosed media may be up to about 5 L, up to about 20 L, up to about 1,000 L, up to about 3,000 L in volume, and up to an industrial scale, such as 30,000 L in volume.

### EXAMPLES

### Example 1: Identification of Factors Determining Yield, Sporulation, and Cargo Protein Activity

Experiments were performed to develop a cost-effective fermentation method for recombinant exosporium-producing Bacillus cells that express a protein or peptide of interest on their exosporium. Such new fermentation method is capable of yielding high spore titers and protein activity while maintaining high sporulation efficiency. Media prototypes were developed with titer yields ranging from 1×10⁹ spores/mL to 3.5×10⁹ spores/mL, with enhanced protein activity, compared to a base medium, which yielded ˜1×10⁸ spores/mL and significantly lower protein activity. The base medium was derived from a laboratory-scale medium and used a low concentration of yeast extract as its principal source of carbon and nitrogen (the “Base Medium”).

Initial experiments were designed to elucidate the main factors or combinations of factors driving the key responses such as spore titer, sporulation rate, and protein activity. Separate experiments with standard lab media showed that brain heart infusion (BHI) broth+0.5% glycerol; Lauria-Bertani (LB) broth, containing tryptone, yeast extract and sodium chloride; succinate nutrient agar (SNA); and tryptic soy broth (TSB), containing casein digest, soybean meal digest, dextrose, sodium chloride and dipotassium phosphate; did not produce improved results compared with the Base Medium. Further, while experimental media without yeast extract produced bacterial growth, the sporulation rates varied substantially. These results demonstrate that multiple factors contribute to media performance.

Data on fermentation outcomes was compiled, and machine learning models were trained to predict process yield. Based on several sets of fermentation data, models were developed to assess the relative contribution of several fermentation parameters including each of the media components. Several factors (temperature, harvest time, yeast extract, total carbohydrate, total carbon plus nitrogen, and total solids) accounted for over 80% of the variation in yield. Further analysis indicated that the major factors influencing sporulation efficiency are related to interactions between carbon and nitrogen sources.

### Example 2: Identification of Novel Media Candidates

Experiments were designed to unravel the main factors and combinations of factors driving the key responses, i.e., spore titer, sporulation rate and cargo protein activity, exhibited in a model system as fluorescence. Fermentations were carried out using recombinant Bacillus thuringiensis strain Bt013A, which had been engineered to display on its exosporium a fluorescent protein, tdTomato, which could be detected to assess protein activity. Briefly, to construct a Bacillus cereus family member displaying the tdTomato fluorescent protein (the “tdTomato Strain”) the pSUPER plasmid was generated through fusion of the pUC57 plasmid (containing an ampicillin resistance cassette and a ColE1 origin of replication) with the pBC16-1 plasmid from Bacillus cereus (containing a tetracycline resistance gene, repU replication gene and oriU origin of replication). This 5.8 kb plasmid can replicate in both E. coli and Bacillus spp. and can be selected by conferring resistance to β-lactam antibiotics in E. coli and resistance to tetracycline in Bacillus spp. The basal pSUPER plasmid was modified by insertion of a PCR-generated fragment that fused the BclA promoter (SEQ ID NO: 1), a start codon, amino acids 20-35 of BclA (amino acids 20-35 of SEQ ID NO: 2) and an alanine linker sequence in frame with SEQ ID NO: 3 resulting in a plasmid termed pSUPER-BclA 20-35-SEQ ID NO: 3. This construct was transformed into E. coli and plated on Lysogeny broth plates plus ampicillin (100 μg/mL) to obtain single colonies. Individual colonies were used to inoculate Lysogeny broth plus ampicillin and incubated overnight at 37° C., 300 rpm. Plasmids from resulting cultures were extracted using a commercial plasmid purification kit. DNA concentrations of these plasmid extracts were determined via spectrophotometry, and obtained plasmids subjected to analytical digests with appropriate combinations of restriction enzymes. The resulting digestion patterns were visualized by agarose gel electrophoresis to investigate plasmid size and presence of distinct plasmid features. Relevant sections of the purified pSUPER derivatives were further investigated by Sanger sequencing. Verified pSUPER-BclA 20-35-SEQ ID NO: 3 plasmids were introduced by electroporation into Bacillus thuringiensis BT013A. Single transformed colonies were isolated by plating on nutrient broth plates containing tetracycline (10 μg/mL). Individual positive colonies were used to inoculate brain heart infusion broth containing tetracycline (10 μg/mL) and incubated overnight at 30° C., 300 rpm. Genomic DNA of resulting cultures was purified and relevant sections of the pSUPER-BclA 20-35-SEQ ID NO: 3 plasmid were re-sequenced to confirm genetic purity of the cloned sequences. Verified colonies were grown overnight in brain heart infusion broth with 10 μg/mL tetracycline and induced to sporulate through incubation in the Base Medium or an experimental medium at 30° C. for 48 hours.

Process conditions were as follows: inoculate production medium with seed medium culture at an optical density of ˜1.0 Au, and grow at 30° C. for 48 hrs-64 hrs (harvest time dependent on sporulation rates). Experiments at a microreactor scale were not pH controlled due to equipment limitation, but were pH controlled (by addition of acid and base) when scaled up to 5 L bioreactors. From these experiments, it became clear that enrichment of the Base Medium with additional carbon and nitrogen ingredients was necessary. Once a selected set of key parameters were identified, further experiments were designed to find the suitable levels of ingredients, which led to the M0-M5 media prototypes. To confirm and compare results, experiments were carried out to test the performance of the tdTomato Strain with three leading media prototypes M0, M2 & M5 along with the Base Medium, focusing on spore titer and fluorescence. Performance was evaluated using the whole broth to test spore titer with a basic hemocytometer method and tdTomato fluorescence with a fluorescence microplate reader (Ex: 551 nm; Em: 584 nm). Results are reported in Example 4, below. Table 2 shows the composition of media prototypes M0-M5 which provided higher titer yields and enhanced cargo protein activity (fluorescence) compared to the Base Medium.

TABLE 2
Composition of Novel Media Compositions

| | M0 | M1 | M2 | M3 | M4 | M5 | |
|---|---|---|---|---|---|---|---|
| | Media | Media | Media | Media | Media | Media | |
| | g/L | g/L | g/L | g/L | g/L | g/L | OM3 |
| Yeast Extract | 25 | 10 | 10 | 5 | 3 | 10 | 5 |
| Soy Flour | 0 | 10 | 15 | 20 | 20 | 30 | 16.5 |
| Cotton Seed | 0 | 0 | 0 | 5 | 2 | 0 | 0 |
| Corn Steep | 0 | 0 | 0 | 5 | 2 | 0 | 0 |
| Glucose | 25 | 25 | 30 | 25 | 25 | 30 | 30 |
| CaCl₂*2H₂O | 0.025 | 0.025 | 0.025-0.375 | 0.025 | 0.025 | 0.725 | 0.02 |
| K₂HPO₄ | 1-3 | 1-3 | 1-3 | 1-3 | 1-3 | 1-3 | 2.5 |
| MgSO₄*7H₂O | 0.2 | 0.2 | 0.2-0.45 | 0.2 | 0.2 | 0.7 | 1.1 |
| (NH₄)₂SO₄ | 1 | 1 | 1 | 1 | 1 | 1 | 5 |
| KH₂PO₄ | 0.8 | 0.8 | 0.8 | 0.8 | 0.8 | 0.8 | 2.6 |
| Na₂HPO₄ | 0.702 | | | | | | |
| Assorted Salts | 0.0355 | 0.0355 | 0.10975 | 0.0355 | 0.0355 | 0.061 | Only |
| (Micronutrients) | | | | | | | ZnSO₄*7H₂O: |
| | | | | | | | 0.03 |

### Example 3: Buffering of Novel Media

An experiment was then carried out to test the performance of the tdTomato strain with the six leading media prototypes M0, M1, M2, M3, M4, and M5 compared to the Base Medium. In Examples 3 and 4, for M2, CaCl₂*2H₂O and MgSO₄*7H₂O were used at the lower end of the concentration ranges provided in Table 2; namely, 0.025 g/L and 0.02 g/L, respectively. Because the microreactors in which the experiment was conducted did not have pH control, the pH was observed to drop to very low levels in media M1, M2, M3, M4, and M5. The Base Media sporulated well and led to expected spore titers (1×10⁸ spores/mL). tdTomato fluorescence from the spores was visible and was confirmed via a plate reader.

To confirm that the pH drop was indeed a factor leading to decreased growth and/or low sporulation in some of the novel media, experiments were performed in the pH-controlled environment of 5 L bioreactors. The strain grew well with several novel media (M0, M2, M5) in a pH-controlled environment with higher acid and base consumption than in fermentation runs using the Base Medium. tdTomato protein production was visible and fluorescence was measured via a plate reader. Protein concentration per spore appeared to be higher in the media prototypes, as compared to the Base Medium.

In order to reduce variations in pH in a non-pH-controlled environment, such as a microreactor or a shake flask, experiments were designed to test buffers of different strengths in the novel media as shown in Table 3. The optimization of the buffer appeared to solve the pH variation issues. 1×B media resulted in larger pH variations, while 2.5×B media provided variations within acceptable ranges. 4×B and 6×B media significantly decreased variations. The terms 1×, 2.5×, 4× and 6× refer to buffering capacity calculations and not to differences in volume of buffer components. It should be noted that such buffers are not required in fermenters that allow for monitoring and adjustment of pH during fermentation through addition of acid or base, as needed.

TABLE 3
Buffering Systems Used in Novel Media

| | Buffer Capacities (g/L) | | | |
|---|---|---|---|---|
| | 1B | 2.5B | 4B | 6B |
| K₂HPO₄ | 2.5 | 8 | 15 | 20 |
| KH₂PO₄ | 0.83 | 1.8 | 2 | 5 |

### Example 4: Spore Titer and Cargo Protein Activity with Novel Media

The effects of a pH-controlled environment were further investigated in 5 L bioreactors, and the performance of the tdTomato Strain was tested with three of the new media prototypes M0, M2, and M5, and with the Base Medium. Phosphate levels were as follows: K₂HPO₄ at 1 g/L and KH₂PO₄ at 0.8 g/L. In these experiments acid and base addition was used to control pH rather than the buffering system described in Example 3. Spore titer, percent sporulation, and fluorescence are shown in Table 4.

TABLE 4
Spore Titer, Sporulation Rate, and Fluorescence
Using Base Medium or Novel Media M0, M2, or M5

| | Base | | | |
|---|---|---|---|---|
| | Medium | M0 | M2 | M5 |
| CFU: Spores/mL | 9.6 × 10⁷ | 1 × 10⁹ | 2.5 × 10⁹ | 3.5 × 10⁹ |
| (hemocytometer) | | | | |
| % Sporulation | 94.5 | 94.06 | 93.31 | 72.73 |
| Fluorescence | 2,421 | 66,768 | 131,901 | 49,720 |

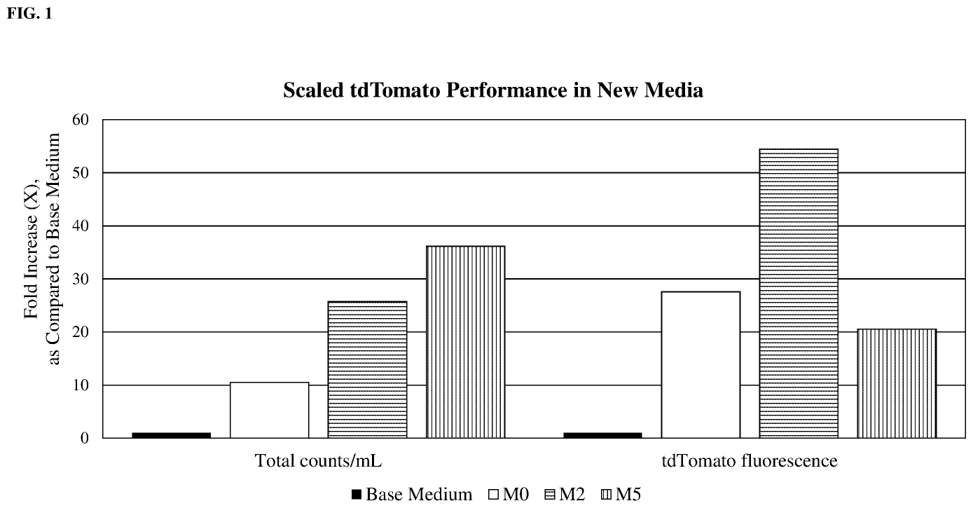

Scaled tdTomato performance in Base Medium and novel media prototypes M0, M2, and M5 are shown in FIG. 1. Fluorescence was higher with novel media in these experiments. Note that % sporulation of M5 was not as high as for M0 and M2 due to a malfunction of the pH probe that led to suboptimal pH and lower sporulation for that specific M5 batch. (In other experiments, typically fermentation in M5 media achieved sporulation of 95% or above.) For M2, scaled performance shows that the fold increase in fluorescence was greater than the fold increase in cells, indicating that each spore has a higher level of displayed tdTomato protein when fermented in the improved method as compared to the method using Base Medium.

### Example 5: Performance of Novel Media with Other Bacillus Strains

The above experiments were conducted with recombinant exosporium-producing Bacillus thuringiensis cells that express a protein or peptide of interest on their exosporium through fusion to a targeting sequence. The performance of media M2 also was investigated with such recombinant exosporium-producing Bacillus cells from several other exosporium-producing Bacillus species. In this Example 5, for M2, CaCl₂*2H₂O and MgSO₄*7H₂O were used at the higher end of the concentration ranges provided in Table 2; namely, 0.375 g/L and 0.45 g/L, respectively.

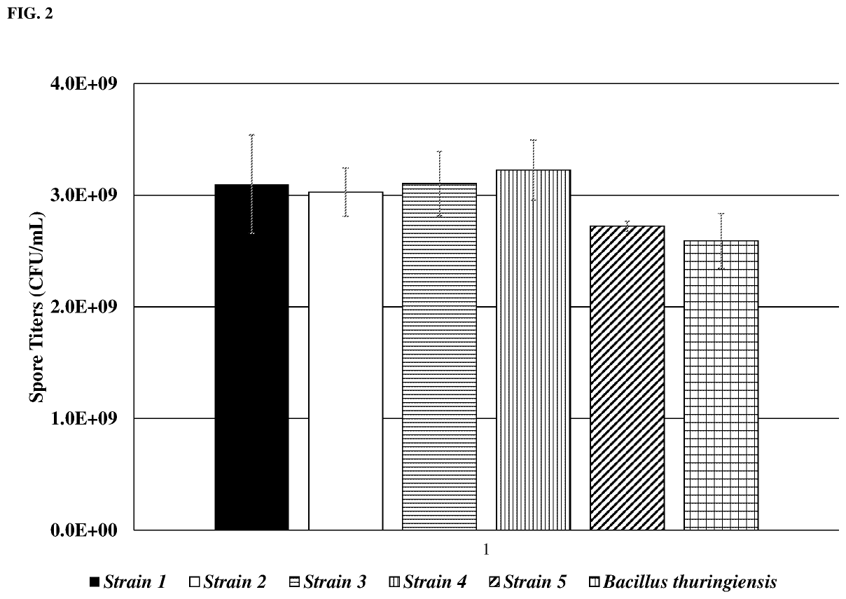

As shown in FIG. 2, spore titers exceeded 2×10⁹ CFU/mL in each strain tested. This represents a substantial increase over spore titers of 1×10⁸ observed using the Base Medium.

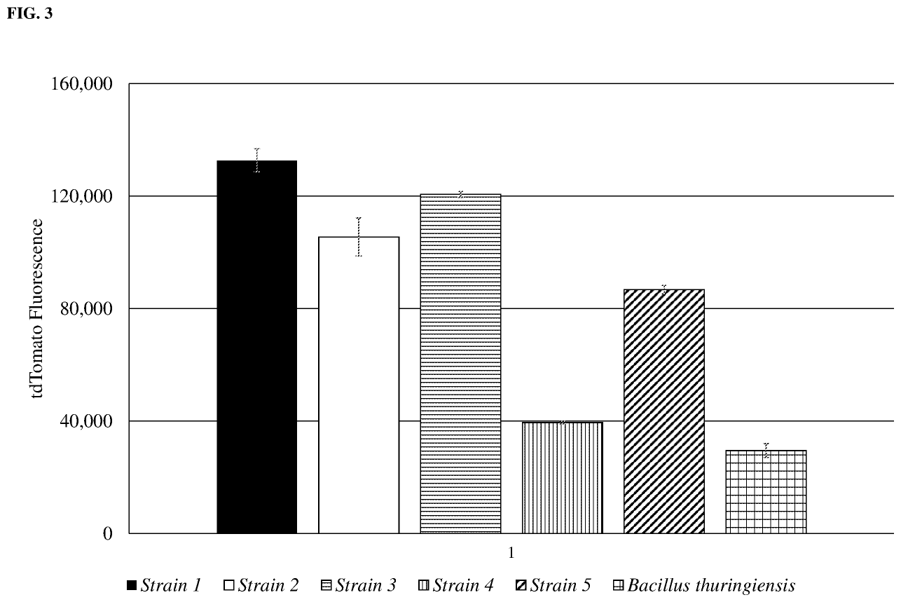

As shown in FIG. 3, strong tdTomato fluorescence was observed using novel media M2 in Strains #1-#4, indicating robust protein expression with the use of the novel media.

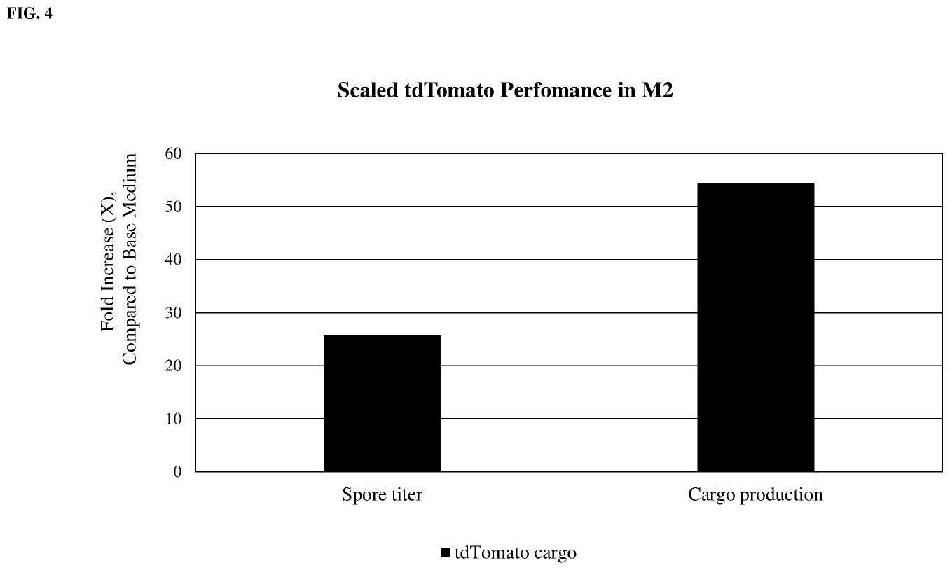

As shown in FIG. 4, improved medium M2 produced substantial increases in spore titer and protein production when used with bacterial systems displaying the tdTomato fluorescent protein. Similar to the results in Example 4, the fold increase for fluorescence was higher than that for spore titer, indicating that each spore has a higher level of displayed protein than when the cells are fermented in the Base Medium.

### Example 6: Spore Titer and Cargo Protein Activity of tdTomato with Novel Media Prototypes M2 and OM3

Experiments were carried out to test the performance of the tdTomato Strain described in Example 2 with novel media prototypes M2 and OM3 compared to the Base Medium. Table 2 shows the composition of media M2 and OM3 which provided higher titer yields and enhanced cargo protein activity (fluorescence) compared to the Base Medium. For M2, CaCl₂*2H₂O and MgSO₄*7H₂O were used at the lower end of the concentration ranges provided in Table 2; namely, 0.025 g/L and 0.02 g/L, respectively.

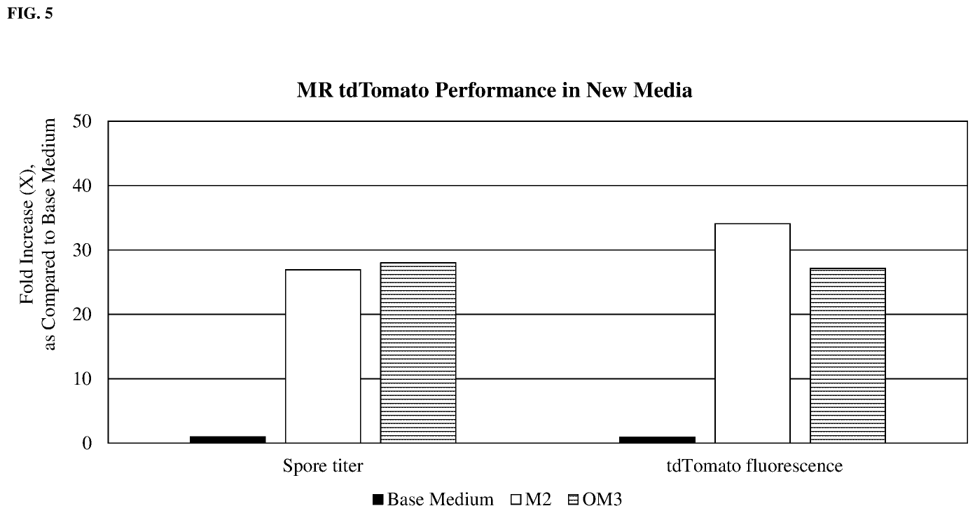

tdTomato Strain was fermented in Base Medium, novel media M2 and novel media OM3 at the microreactor scale. Spore titer and fluorescence were evaluated as in Example 2, and scaled tdTomato performance is shown in FIG. 5. Spore titer and percent sporulation are shown in Table 5.

TABLE 5
Spore Titer, Sporulation Rate, and Fluorescence Using Base
Medium or Novel Media M2 or OM3 at Microreactor Scale

| | Base | | |
|---|---|---|---|
| | Medium | M2 | OM3 |
| CFU: Spores/mL | 1.5 × 10⁸ | 4.0 × 10⁹ | 4.2 × 10⁹ |
| (hemocytometer) | | | |
| % Sporulation | 92.4 | 94.5 | 97.1 |

### Example 7: Construction of a Bacillus cereus Family Member Displaying a Serine Protease or Serine Protease Variant

Further experiments were carried out using a recombinant Bacillus thuringiensis strain Bt013A which had been engineered to display on its exosporium a serine protease (Sep1 variant), which could assayed for protein activity. Briefly, a Bacillus cereus family member displaying the Sep1 variant protein and having an ExsY knockout (the “Sep1 Strain”) was constructed.

To construct a Bacillus cereus family member displaying a Sep1 variant, the pSUPER plasmid was generated through fusion of the pUC57 plasmid (containing an ampicillin resistance cassette and a ColE1 origin of replication) with the pBC16-1 plasmid from Bacillus cereus (containing a tetracycline resistance gene, repU replication gene and oriU origin of replication). This 5.8 kb plasmid can replicate in both E. coli and Bacillus spp. and can be selected by conferring resistance to ρ3-lactam antibiotics in E. coli and resistance to tetracycline in Bacillus spp. The basal pSUPER plasmid was modified by insertion of a PCR-generated fragment that fused a promoter, a start codon, a targeting sequence, and an alanine linker sequence in frame with SEQ ID NO: 7, encoding the Sep1 variant, resulting in pSUPER plasmids. This construct was transformed into E. coli and plated on Lysogeny broth plates plus ampicillin (100 μg/mL) to obtain single colonies. Individual colonies were used to inoculate Lysogeny broth plus ampicillin and incubated overnight at 37° C., 300 rpm. Plasmids from resulting cultures were extracted using a commercial plasmid purification kit. DNA concentrations of these plasmid extracts were determined via spectrophotometry, and obtained plasmids subjected to analytical digests with appropriate combinations of restriction enzymes. The resulting digestion patterns were visualized by agarose gel electrophoresis to investigate plasmid size and presence of distinct plasmid features. Relevant sections, such as the Sep1 variant expression cassette, of the purified pSUPER derivatives were further investigated by Sanger sequencing.

pSUPER plasmids, verified as described above, were introduced by electroporation into Bacillus thuringiensis BT013A (Accession No. NRRL B-50924). Single transformed colonies were isolated by plating on nutrient broth plates containing tetracycline (10 μg/mL). Individual positive colonies were used to inoculate brain heart infusion broth containing tetracycline (10 μg/mL) and incubated overnight at 30° C., 300 rpm. Genomic DNA of resulting cultures was purified and relevant sections of the pSUPER plasmid were re-sequenced to confirm genetic purity of the cloned sequences. Verified colonies were grown overnight in brain heart infusion broth with 10 μg/mL tetracycline and induced to sporulate through incubation in a yeast extract-based media at 30° C. for 48 hours.

To make exsY knockout (KO) mutant strains of Bacillus thuringiensis BT013A, the plasmid pKOKI shuttle and integration vector was constructed that contained the pUC57 backbone, which is able to replicate in E. coli, as well as the origin of replication and the erythromycin resistance cassette from pE194. This construct is able to replicate in both E. coli and Bacillus spp. A construct was made that contained the 1 kb DNA region that corresponded to the upstream region of the exsY gene and a 1 kb region that corresponded to the downstream region of the gene exsY, both of which were PCR amplified from Bacillus thuringiensis BT013A. For each construct, the two 1 kb regions were then spliced together using homologous recombination with overlapping regions to each other and with the pKOKI plasmid, respectively. This plasmid construct was verified by digestion and DNA sequencing. Clones were screened for erythromycin resistance.

Clones were passaged under high temperature (40° C.) in brain heart infusion broth. Individual colonies were toothpicked onto LB agar plates containing erythromycin 5 μg/mL, grown at 30° C., and screened for the presence of the pKOKI plasmid integrated into the chromosome by colony PCR. Colonies that had an integration event were continued through passaging to screen for single colonies that lost erythromycin resistance (signifying loss of the plasmid by recombination and removal of the exsY gene). Verified deletions were confirmed by PCR amplification and sequencing of the target region of the chromosome. Finally, the PCR-amplified, circularized pBC section of the pSUPER plasmids (described above) was transformed into this exsY mutant strain of BT013A. The resulting strain is a Bacillus cereus family member displaying the Sep1 variant protein and having an exsY knockout (the “Sep1 Strain”).

For each exsYKO mutant expressing the serine protease variant, an overnight culture was grown in BHI media at 30° C., 300 rpm, in baffled flasks with antibiotic selection. One milliliter of this overnight culture was inoculated into a yeast extract-based media (50 mL) in a baffled flask and grown at 30° C. for 2 days. An aliquot of spores was removed and the spores were agitated by vortexing. The spores were collected via centrifugation at 8,000×g for 10 minutes, and supernatant containing the exosporium fragments was filtered through a 0.22 μm filter to remove any residual spores. No spores were found in the filtrate.

### Example 8: Spore Titer and Cargo Protein Activity of the Sep1 Strain with Novel Media Prototypes M2 and OM3

The Sep1 Strain was produced via fermentation in flasks or 20 L fermenters. Briefly, an overnight brain heart infusion seed flask with 10 μg/mL tetracycline of the Sep1 Strain was inoculated into 1 L shake flasks or 20 L fermenters of M2 or OM3 media and cultured from 48 to 72 hrs at 30° C. to produce >90% endospores. For M2, CaCl₂*2H₂O and MgSO₄*7H₂O were used at the lower end of the concentration ranges provided in Table 2; namely, 0.025 g/L and 0.02 g/L, respectively. For this study, the harvested whole cell broth constituted the final product. Exosporium fragments were not collected prior to the analyses described below.

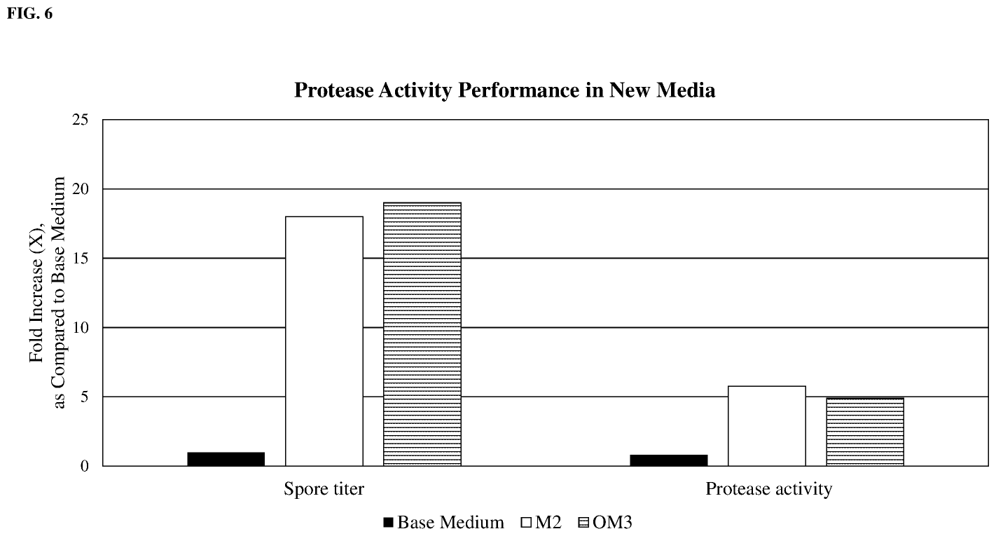

Protease activity was measured at fermentation harvest (no downstream processing was performed). Enzyme activity was determine using synthetic peptide substrate (Ala-Ala-Pro-Phe). The peptide substrate is fused with nitro phenyl at the C-terminus and a succinyl group at the N-terminus. The peptide shows absorbance maxima at 320 nm before protease cleavage and shifts to 390 nm following the cleavage. The assay mixture consisted of 2.5 mg/mL peptide substrate in 240 μL of 50 mM Hepes buffer pH 7.5, containing 5 mM CaCl₂. The substrate and the buffer were pre-incubated at room temperature, followed by the addition of 25 μL of the enzyme solution. Protease activity of the Sep1 Strain in Base Medium or novel media prototypes M2 or OM3 are shown in FIG. 6.

Spore titers were measured via hemocytometer counts in the fermentation whole broth. Results are shown in Table 6.

TABLE 6
Spore Titer and Sporulation Rate Using
Base Medium or Novel Media M2 or OM3

| | Base | | |
|---|---|---|---|
| | Medium | M2 | OM3 |
| CFU: Spores/mL | 2.0 × 10⁸ | 3.6 × 10⁹ | 3.8 × 10⁹ |
| (hemocytometer) | | | |
| % Sporulation | Not | 97.6 | 95.4 |
| | performed | | |

1. (canceled)

2. A method of producing a fermentation product from recombinant exosporium-producing Bacillus cells that express a fusion protein, comprising:
culturing the recombinant exosporium-producing Bacillus cells that express a fusion protein in a medium comprising
i) yeast extract at a concentration of about 3 g/L to about 20 g/L;
ii) glucose at a concentration of up to about 35 g/L;
iii) soy flour at a concentration of up to about 50 g/L; and
iv) a source of Ca²⁺ ion;
wherein the fusion protein comprises a protein or peptide of interest and a targeting sequence, exosporium protein, or exosporium protein fragment.

3. (canceled)

4. The method of claim 2, wherein the medium further comprises a source of Mg²⁺ ion.

5. (canceled)

6. The method of claim 2, wherein the medium further comprises cotton seed flour at a concentration of up to about 10 g/L.

7. The method of claim 2, wherein the medium further comprises corn steep liquor at a concentration of up to about 10 g/L.

8. The method of claim 2, further comprising maintaining pH of 6-8 during the culturing.

9. The method of claim 8, wherein maintaining pH is accomplished by addition of acid or base.

10. The method of claim 8, wherein the medium further comprises a buffer.

11. The method of claim 10, wherein the buffer is K₂HPO₄ and KH₂PO₄.

12. The method of claim 11, wherein K₂HPO₄ is present at a concentration of at least 1 g/L and KH₂PO₄ is present at a concentration of at least 0.8 g/L.

13. The method of claim 2, wherein culturing occurs at 25° C.-35° C.

14. The method of claim 2, wherein culturing occurs for up to 50 hours.

15. The method of claim 2, wherein culturing occurs until sporulation of the Bacillus cells is at least 90% complete.

16. The method of claim 2, wherein the culturing results in a fermentation broth having a spore titer of at least 1×10⁹ spores/mL.

17. The method of claim 2, wherein the medium comprises one or more sources of carbon having a total concentration of at least 20 g/L.

18. The method of claim 17, wherein the medium comprises one or more sources of nitrogen having a total concentration of at least 3 g/L.

19. The method of claim 18, wherein the concentration, when combined, of the one or more sources of carbon and the one or more sources of nitrogen is at least 15 g/L.

20. The method of claim 2, wherein the medium comprises
a) yeast extract at a concentration of about 3 g/L to about 25 g/L;
b) glucose at a concentration of up to about 30 g/L
c) soy flour at a concentration of up to about 30 g/L;
d) a buffer comprising K₂HPO₄ at a concentration of about 0.5 g/L to about 5 g/L and KH₂PO₄ at a concentration of about 0.1 g/L to about 5 g/L;
e) CaCl₂*2H₂O at a concentration of about 0.010 g/L to about 1 g/L; and
f) MgSO₄*7H₂O at a concentration of about 0.1 g/L to about 1.5 g/L.

21. The method of claim 20, wherein the medium comprises
a) yeast extract at a concentration of about 5 g/L to about 15 g/L;
b) glucose at a concentration of about 20 g/L to about 35 g/L;
c) soy flour at a concentration of about 10 g/L to about 30 g/L;
d) a buffer comprising K₂HPO₄ at a concentration of about 1 g/L to about 5 g/L and KH₂PO₄ at a concentration of about 0.5 g/L to about 2 g/L;
e) CaCl₂*2H₂O at a concentration of about 0.015 g/L to about 0.80 g/L; and
f) MgSO₄*7H₂O at a concentration of about 0.10 g/L to about 0.80 g/L.

22. The method of claim 20, wherein the medium comprises
a) yeast extract at a concentration of about 10 g/L to about 15 g/L;
b) glucose at a concentration of about 25 g/L to about 30 g/L;
c) soy flour at a concentration of about 15 g/L to about 20 g/L;
d) a buffer comprising K₂HPO₄ at a concentration of about 1 g/L to about 3 g/L and KH₂PO₄ at a concentration of about 0.5 g/L to about 1 g/L;
e) CaCl₂*2H₂O at a concentration of about 0.02 g/L to about 0.4 g/L; and
f) MgSO₄*7H₂O at a concentration of about 0.2 g/L to about 0.5 g/L.

23. The method of claim 2, wherein the protein or peptide of interest is selected from the group consisting of a plant growth stimulating protein or peptide, a protein or peptide that protects a plant from a pathogen, and an insecticidal protein or peptide.

24. The method of claim 2, wherein
the targeting sequence, exosporium protein or exosporium protein fragment comprises the sequence X₁-X₂-X₃-X₄-X₅-X₆-X₇-X₈-X₉-X₁₀-X₁₁-X₁₂-X₁₃-X₁₄-X₁₅-X₁₆, wherein:
X₁ is any amino acid or absent;
X₂ is phenylalanine (F), leucine (L), isoleucine (I), or methionine (M);
X₃ is any amino acid;
X₄ is proline (P) or serine (S);
X₅ is any amino acid;
X₆ is leucine (L), asparagine (N), serine (S), or isoleucine (I);
X₇ is valine (V) or isoleucine (I);
X₈ is glycine (G);
X₉ is proline (P);
X₁₀ is threonine (T) or proline (P);
X₁₁ is leucine (L) or phenylalanine (F);
X₁₂ is proline (P);
X₁₃ is any amino acid;
X₁₄ is any amino acid;
X₁₅ is proline (P), glutamine (Q), or threonine (T); and
X₁₆ is proline (P), threonine (T), or serine (S).

25. The method of claim 2, wherein the targeting sequence, exosporium protein or exosporium protein fragment comprises:
an amino acid sequence having at least about 43% identity with amino acids 20-35 of SEQ ID NO: 1, wherein the identity with amino acids 25-35 is at least about 54%;
a targeting sequence comprising amino acids 1-35 of SEQ ID NO: 1;
a targeting sequence comprising amino acids 20-35 of SEQ ID NO: 1;
a targeting sequence comprising amino acids 22-31 of SEQ ID NO: 1;
a targeting sequence comprising amino acids 22-33 of SEQ ID NO: 1;
a targeting sequence comprising amino acids 20-31 of SEQ ID NO: 1;
a targeting sequence comprising SEQ ID NO: 1; or
an exosporium protein comprising an amino acid sequence having at least 85% identity with SEQ ID NO: 2.

26. The method of claim 2, wherein the exosporium-producing Bacillus cells are cells of a Bacillus cereus family member.

27-34. (canceled)

35. A fermentation broth or fermentation product produced by the method of claim 20.

36-37. (canceled)


SEQUENCE LISTING (10 sequences)

SEQ ID NO: 1 - DNA, 300 bp, Bacillus cereus
atcgatggaacctgtatcaaccactataatttcatccacaattttttcaactgagtctaaacaacgggctattgtcttctcctcatctcgaacaatcatacataaactaattgtaattccttgcttgttcaacataatcaccctcttccaaatcaatcatatgttatacatatactaaactttccatttttttaaattgttcaagtagtttaagatttcttttcaataattcaaatgtccgtgtcattttctttcggttttgcatctactatataatgaacgctttatggaggtgaattt

SEQ ID NO: 2 - protein, 41 aa, Bacillus anthracis
MSNNNYSNGLNPDESLSASAFDPNLVGPTLPPIPPFTLPTG

SEQ ID NO: 3 - protein, 476 aa, Artificial Sequence
  tdTomato fluorescent protein
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYK

SEQ ID NO: 4 - protein, 332 aa, Bacillus anthracis
MSNNNYSNGLNPDESLSASAFDPNLVGPTLPPIPPFTLPTGPTGPFTTGPTGPTGPTGPTGPTGPTGPTGPTGDTGTTGPTGPTGPTGPTGPTGPTGPTGPTGPTGFTPTGPTGPTGPTGDTGTTGPTGPTGPTGPTGPTGDTGTTGPTGPTGPTGPTGPTGPTGPTFTGPTGPTGPTGATGLTGPTGPTGPSGLGLPAGLYAFNSGGISLDLGINDPVPFNTVGSQFFTGTAISQLDADTFVISETGFYKITVIANTATASVLGGLTIQVNGVPVPGTGSSLISLGAPFTIVIQAITQITTTPSLVEVIVTGLGLSLALGTSASIIIEKVA

SEQ ID NO: 5 - protein, 321 aa, Bacillus firmus
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCAAGNEGDGDDSTDEFAYPGCYNEVISVGAINLERDSSDFTNSHNEIDLVAPGEGILSTFLNGKYATLSGTSMAAPHVSGALALIKDFANRQFERKLSEPELYAQLIRRTVPLGNSPKLEGNGLVYLTVPDHLAGIFDQELKSTVLNAI

SEQ ID NO: 6 - protein, 321 aa, Bacillus firmus
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENDAGVIGVAPEADLLIVKVLNKDGSGQYEWIIKGIHYAIEQNADIISMSLGGPADVPELHDAIKAAVNKNILVVCAAGNEGDGDDSTDEFAYPGCYNEVISVGAINLERDSSEFTNSHNEIDLVAPGEGILSTFLNGKYATLSGTSMAAPHVSGALALIKEFANRQFERKLSEPELYAQLIRRTVPLGNSPKLEGNGLVYLTVPDHLAGIFDQEFKSTVLNAI

SEQ ID NO: 7 - protein, 261 aa, Bacillus firmus
MEQYVRVIPYKVIQQEENVKEVPKGVELIQAPKVWSETKGKGIKIAVLDTGCDISHPDLKDRVTGGRNFTDDDNSDPNSFKDYNGHGTHVAGTIAAYENNAGVIGVAPEAELLIVKVLNKDGSGQYEWIIKGIHYAIEQNADIISMSLGGPADVPELHDAIKAAVNKNILVVCAAGNEGDSGTSMAAPHVSGALALIKDFANRQFERKLSEPELYAQLIRRTVPLGNSPKLEGNGLVYLTVPDHLAGIFDQELKSTVLNAI

SEQ ID NO: 8 - protein, 499 aa, Bacillus subtilis
MKRSISIFITCLLITLLTMGGMIASPASAAGTKTPVAKNGQLSIKGTQLVNRDGKAVQLKGISSHGLQWYGEYVNKDSLKWLRDDWGITVFRAAMYTADGGYIDNPSVKNKVKEAVEAAKELGIYVIIDWHILNDGNPNQNKEKAKEFFKEMSSLYGNTPNVIYEIANEPNGDVNWKRDIKPYAEEVISVIRKNDPDNIIIVGTGTWSQDVNDAADDQLKDANVMYALHFYAGTHGQFLRDKANYALSKGAPIFVTEWGTSDASGNGGVFLDQSREWLKYLDSKTISWVNWNLSDKQESSSALKPGASKTGGWRLSDLSASGTFVRENILGTKDSTKDIPETPSKDKPTQENGISVQYRAGDGSMNSNQIRPQLQIKNNGNTTVDLKDVTARYWYKAKNKGQNFDCDYAQIGCGNVTHKFVTLHKPKQGADTYLELGFKNGTLAPGASTGNIQLRLHNDDWSNYAQSGDYSFFKSNTFKTTKKITLYDQGKLIWGTEPN

SEQ ID NO: 9 - protein, 283 aa, Bacillus thuringiensis
MKKKVLALAAAITLVAPLQSVAFAHENDGGQRFGVIPRWSAEDKHKEGVNSHLWIVNRAIDIMSRNTTLVKQDRVALLNEWRTELENGIYAADYENPYYDNSTFASHFYDPDNGKTYIPYAKQAKETGAKYFKLAGESYKNKDMQQAFFYLGLSLHYLGDVNQPMHAANFTNLSYPQGFHSKYENFVDTIKDNYKVTDGNGYWNWKGTNPEDWIHGAAVVAKQDYAGIVNDNTKDWFVRAAVSQEYADKWRAEVTPMTGKRLMDAQRVTAGYIQLWFDTYGDR

SEQ ID NO: 10 - protein, 244 aa, Bacillus subtilis
LEAGLNKDQKRRAEQLTSIFENGTTEIQYGYVERLDDGRGYTCGRAGFTTATGDALEVVEVYTKAVPNNKLKKYLPELRRLAKEESDDTSNLKGFASAWKSLANDKEFRAAQDKVNDHLYYQPAMKRSDNAGLKTALARAVMYDTVIQHGDGDDPDSFYALIKRTNKKAGGSPKDGIDEKKWLNKFLDVRYDDLMNPANHDTRDEWRESVARVDVLRSIAKENNYNLNGPIHVRSNEYGNFVIK